(19) United States
(12) Patent Application Publication
(10) Pub. No.: US 2003/0157714 A1
(43) Pub. Date: Aug. 21, 2003

(54) Apparatus for collecting detached cells, method of the same, and program for the same

(75) Inventors: Ryota Umegaki, Osaka (JP); Masahiro Kino-Oka, Osaka (JP); Masahito Taya, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Masahito Taya, Toyonaka-shi (JP)

(21) Appl. No.: 10/224,497

(22) Filed: Aug. 21, 2002

(30) Foreign Application Priority Data
Feb. 20, 2002 (JP) 2002-043550

(51) Int. Cl. C12M 3/00; C12N 5/00

(52) U.S. Cl. 435/378; 435/3; 435/286.5; 435/308.1

(57) ABSTRACT

A control device of a detached cells collection apparatus first closes an exhaust pipe and opens an injection pipe to inject a predetermined quantity of a trypsin solution into a culture chamber, starts counting trypsin processing time, and inclines a support table when the counted trypsin processing time reaches a preset time period. The control device simultaneously opens the exhaust pipe and makes the trypsin solution including detached cells in the culture chamber flown through the exhaust pipe to a collection chamber. On completion of such collection, the support table is returned to its horizontal orientation and a new collection chamber is set in the detached cells collection apparatus. The control device again closes the exhaust pipe and opens the injection pipe to inject the predetermined quantity of the trypsin solution into the culture chamber and resumes counting the trypsin processing time. This series of operations is repeated to collect cells according to the trypsin processing time.

### BACKGROUND OF THE INVENTION

1. Field of the Invention

The present invention relates to a detached cells collection apparatus, a method of collecting detached cells, and a program used for the method.

2. Description of the Prior Art

Lots of techniques have been proposed recently to culture a human tissue in vitro and apply the cultured tissue to a patient's affected area. Especially the techniques for culturing a human skin tissue have reached the commercialized level in various countries. The manufacturing flow of a cultured cell sheet of a human skin tissue, for example, keratinocyte, is described with reference to FIG. 6. The manufacturing process first obtains a small piece of tissue from a non-affected area of a patient or a donor, carries out enzymatic treatment to disperse cells, and isolates keratinocytes (step S1). The process then carries out primary culture of the isolated keratinocytes (step S2). The process inoculates (seeding) the cells proliferated by the primary culture in a plurality of culture chambers (step S3), and changes the culture medium according to the requirements (step S4). The cells adhering to the culture surface are proliferated to gradually increase the cell area on the culture surface to a preset area and reach to a confluent state. The process determines whether or not the total cell area reaches or exceeds a sufficient level (number of cells) required for the product (step S5). When the cell area has not yet reached the sufficient level, the process starts to remove the culture medium, and to detach the cells adhering to the culture surface with a protease, such as trypsin, and collects the detached cells (step S6). Subculture is repeatedly performed until the cell area reaches or exceeds the sufficient level. When the total cell area has reached or exceeded the sufficient level, on the other hand, the process performs three-dimensional culture (multi-layered culture) to build up the tissue (step S7) and delivers a resulting cultured cell sheet to a hospital or another suitable organization for grafting (step S8).

In the detaching process at step S6, the excessively long protease processing time may damage the cells and cause troubles in subsequent culture. It is accordingly preferable to optimize the protease processing time by continuously monitoring the detaching state of cells with elapse of time.

In the detaching process with protease at step S6, the respective cells require different time periods to be detached from. It is accordingly preferable to collect cells detached from the culture surface by taking into account this factor.

### SUMMARY OF THE INVENTION

The object of the present invention is thus to provide a detached cells collection apparatus that adequately collects cells detached from the adhesion surface, a method of collecting detached cells, and a program used for the method.

A first aspect of the present invention provides a detached cells collection apparatus that collect anchorage-dependent cells, which are cultured in a culture chamber and are detached from the culture chamber, the detached cells collection apparatus including: a time counter module that counts an elapsed time since injection of a release agent into the culture chamber with the anchorage-dependent cells adhering thereto; a cell collection module that collects the anchorage-dependent cells in the culture chamber; and a classification-collection control module that actuates the cell collection module to classify cells detached from the culture chamber by the release agent according to the elapsed time counted by the time counter module and collect the classified cells.

The detached cells collection apparatus of the present invention classifies the anchorage-dependent cells, which adhere to the culture chamber and are detached from the culture chamber by means of the release agent added to the culture chamber, based on the time elapsed since the addition of the release agent to the culture chamber, and collects the classified anchorage-dependent cells. This apparatus enables the cells detached from the culture chamber in an early stage by the release agent to be collected separately from the cells detached from the culture chamber after elapse of some time. The sequential collection of the detached cells effectively prevents the cells from being damaged by the release agent.

The ‘anchorage-dependent cells’ represent cells that adhere to a specific face of the culture chamber directly or indirectly via an extracellular matrix, expand their adhesion area, and reach cell division. The anchorage-dependent cells may be any of diverse cells taken from warm-blooded animals including human, mouse, rat, guinea pig, hamster, chicken, rabbit, pig, sheep, cattle, horse, dog, cat, and monkey. The cells taken from such warm-blooded animals may be any of keratinocytes, splenic cells, neurons, glial cells, pancreatic β-cells, mesangium cells, Langerhans cells, epidermic cells, epithelial cells, endothelial cells, fibroblasts, fibrocytes, muscle cells, lipocytes, synovial cells, chondrocytes, osteocytes, osteoblasts, osteoclasts, breast cells, hepatocytes, and interstitial cells, as well as their precursor cells, stem cells, and anchorage-dependent cancer cells and Embryonic Stem cells. The cells may otherwise be transformed cells obtained by inducing foreign genes, which code, for example, erythropoietin, growth hormone, granular colony stimulating factor, insulin, interferon, blood coagulation factor like blood coagulation factor VIII, glucagon, tissue plasminogen activator, dopamine, oncogene, cancer suppressor gene, into these cells to make the genes expressed forcibly or under specific conditions with various promoters. Typical examples of the extracellular matrix include integrin, collagen, elastin, proteoglycan, glycosaminoglycan, and glycoprotein.

The ‘culture chamber’ may be composed of any material as long as the material allows culture of cells. Preferable examples include synthetic resins like polyethylene, polypropylene, polystyrene, polycarbonate, polyvinyl chloride, and polytetrafluoroethylene, hydroxyapatite ceramics, alumina ceramics, and glasses.

In one preferable application, the first detached cells collection apparatus of the present invention may include a release agent injection module that injects a release agent into the culture chamber. The classification-collection control module may actuate the cell collection module according to the elapsed time to recover the practically whole quantity of the release agent and thereby collect the cells detached from the culture chamber. The classification-collection control module may then actuate the release agent injection module to inject a new supply of the release agent to the culture chamber. This arrangement ensures collection of the anchorage-dependent cells taking a substantially identical time to be detached from by the release agent. The procedure of one preferable embodiment repeats the process of recovering the practically whole quantity of the release agent containing the detached cells in the culture chamber and subsequently injecting a new supply of the release agent with elapse of time.

In another preferable application, the first detached cells collection apparatus of the present invention may further include a release agent injection module that injects the release agent into the culture chamber. The classification-collection control module may actuate the cell collection module according to the elapsed time to recover part of the release agent and thereby collect the cells detached from the culture chamber and included in the release agent. The classification-collection control module may subsequently actuate the release agent injection module to inject a new supply of the release agent to the culture chamber. This arrangement ensures collection of the anchorage-dependent cells taking a substantially identical time to be detached from by the release agent, based on the principle similar to fraction collection of liquid chromatography.

In the first detached cells collection apparatus of the present invention, the classification-collection control module may actuate the cell collection module to collect the anchorage-dependent cells detached from the culture chamber as an initial division when the elapsed time counted by the time counter module reaches a preset initial time, and to collect the anchorage-dependent cells detached from the culture chamber after the preset initial time as a main division. When there is a difference in ability (for example, proliferation potency) between the cells detached from the culture chamber in the early stage by the release agent and the cells detached from the culture chamber after elapse of some time, this arrangement enables divisional collection of the cells according to the difference in ability. The release agent may be supplemented after collection of detached cells by the cell collection module.

In the first detached cells collection apparatus of the present invention, the classification-collection control module may actuate the cell collection module to collect the anchorage-dependent cells detached from the culture chamber as an initial division when the elapsed time counted by the time counter module reaches a preset initial time, to collect the anchorage-dependent cells detached from the culture chamber as an intermediate division until the elapsed time reaches a preset latter time, and to collect the anchorage-dependent cells detached from the culture chamber after the preset later time as a last division. There may be a difference in ability or a difference in susceptibility to damage among the cells detached from the culture chamber in an early stage by the release agent, the cells detached from the culture chamber after elapse of an intermediate time period, and the cells detached from the culture chamber after elapse of a longer time period. In such cases, this application enables divisional collection of cells according to their difference in ability or their difference in susceptibility to damage. Although this application performs classification into three groups of elapsed time, initial, intermediate, and last stages, the number of divisions is not restricted to three. The cells may thus be divided into four or more groups of elapsed time according to the requirements. The release agent may be supplemented after collection of detached cells by the cell collection module.

A second aspect of the present invention provides a detached cells collection method for collecting anchorage-dependent cells, which are cultured in a culture chamber and are detached from the culture chamber, the detached cells collection method including: a time counting step that counts an elapsed time since injection of a release agent into the culture chamber with the anchorage-dependent cells adhering thereto; and a collection step that actuates a cell collection module, which is used for collecting cells in the culture chamber, to classify cells detached from the culture chamber by the release agent according to the elapsed time counted in the time counting step and collect the classified cells.

The detached cells collection method of the present invention classifies the anchorage-dependent cells, which adhere to the culture chamber and are detached from the culture chamber by means of the release agent added to the culture chamber, based on the time elapsed since the addition of the release agent to the culture chamber, and collects the classified anchorage-dependent cells. This method enables the cells detached from the culture chamber in the early stage by the release agent to be collected separately from the cells detached from the culture chamber after elapse of some time. The sequential collection of the detached cells effectively prevents the cells from being damaged by the release agent.

In the second detached cells collection method of the present invention, the collection step may actuate the cell collection module according to the elapsed time to recover a substantially whole quantity of the release agent and thereby collect the cells detached from the culture chamber and included in the release agent. The collection step may then actuate a release agent injection module, which is used to inject the release agent into the culture chamber, to inject a new supply of the release agent to the culture chamber.

In the second detached cells collection method of the present invention, the collection step may repeatedly carry out a process of actuating the cell collection module according to the elapsed time to recover a substantially whole quantity of the release agent and thereby collect the cells detached from the culture chamber and included in the release agent, and subsequently actuating the release agent injection module to inject a new supply of the release agent to the culture chamber.

In the second detached cells collection method of the present invention, the collection step may actuate the cell collection module according to the elapsed time to recover part of the release agent and thereby collect the cells detached from the culture chamber and included in the release agent. The collection step may subsequently actuate a release agent injection module, which is used to inject the release agent into the culture chamber, to inject a new supply of the release agent to the culture chamber.

In the second detached cells collection method of the present invention, the collection step may actuate the cell collection module to collect the cells detached from the culture chamber as an initial division when the elapsed time counted by the time counter step reaches a preset initial time, and to collect the cells detached from the culture chamber after the preset initial time as a main division.

In the second detached cells collection method of the present invention, the collection step may actuate the cell collection module to collect the cells detached from the culture chamber as an initial division when the elapsed time counted by the time counter step reaches a preset initial time, to collect the cells detached from the culture chamber as an intermediate division until the elapsed time reaches a preset latter time, and to collect the cells detached from the culture chamber after the preset later time as a last division.

The detached cells collection method for collecting anchorage-dependent cells, which are cultured in a culture chamber and are detached from the culture chamber, may include: a step of actuating a cell collection module, which is used for collecting cells in the culture chamber, to classify cells detached from the culture chamber by a release agent according to an elapsed time since injection of the release agent into the culture chamber with the anchorage-dependent cells adhering thereto, and collect the classified cells.

A third aspect of the present invention provides a program that causes a computer to carry out an operation of collecting anchorage-dependent cells, which are cultured in a culture chamber and are detached from the culture chamber, the program causing the computer to attain:

a time counting function that counts an elapsed time since injection of a release agent into the culture chamber with the anchorage-dependent cells adhering thereto; and

a collection function that actuates a cell collection module, which is used for collecting cells in the culture chamber, to classify cells detached from the culture chamber by the release agent according to the elapsed time counted by the time counting function and collect the classified cells.

The program that causes a computer to carry out an operation of collecting anchorage-dependent cells, which are cultured in a culture chamber and are detached from the culture chamber, may cause the computer to attain the function of: actuating a cell collection module, which is used for collecting cells in the culture chamber, to classify cells detached from the culture chamber by a release agent according to an elapsed time since injection of the release agent into the culture chamber with the anchorage-dependent cells adhering thereto, and collect the classified cells.

Any of these programs may be recorded in a computer readable recording medium (for example, a hard disk, a ROM, FDs, CDs, and DVDs), may be transmitted from one computer to another computer via a communication medium (communication network like the Internet or a LAN), or may be given and received in any other suitable form. The computer executes these programs to attain the same functions and effects as those of the detaching cells collection apparatus of the present invention described above.

### BRIEF DESCRIPTION OF THE DRAWINGS

FIG. 1 schematically illustrates the construction of a detached cells collection apparatus in a first embodiment of the present invention, where a support table is in horizontal orientation;

FIG. 2 schematically illustrates the construction of detached cells collection apparatus of the first embodiment, where the support table is in inclined orientation;

FIG. 3 is a flowchart showing a cell detaching operation program executed in the first embodiment;

FIG. 4 schematically illustrates the construction of another detached cells collection apparatus in a second embodiment of the present invention;

FIG. 5 is a flowchart showing another cell detaching operation program executed in the second embodiment; and

FIG. 6 is a flowchart showing a process of manufacturing a cultured cell sheet.

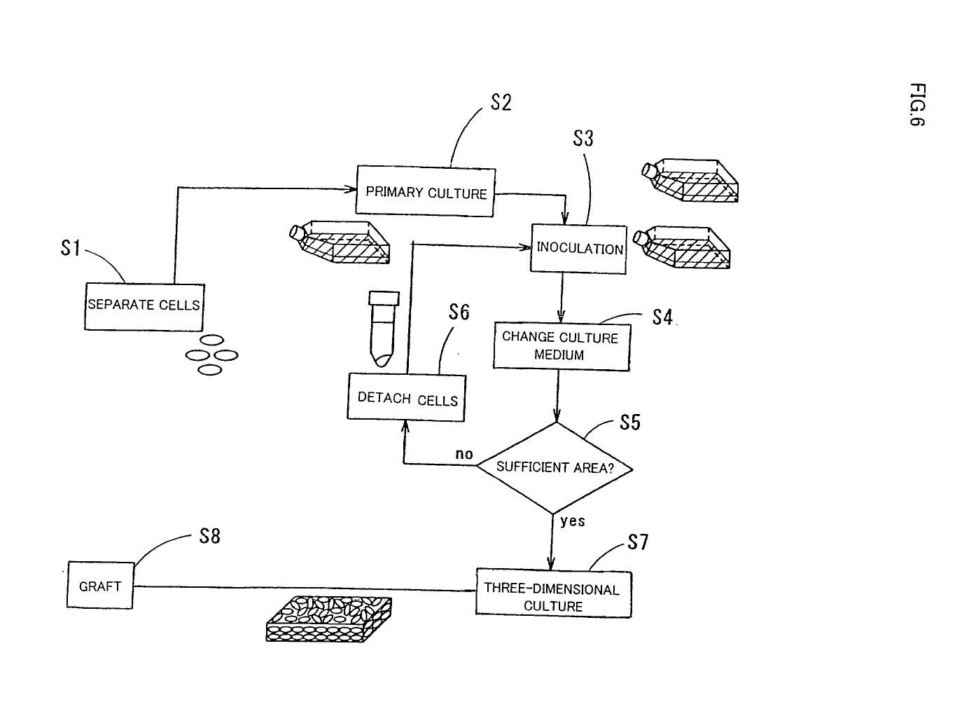

### DESCRIPTION OF THE PREFERRED EMBODIMENTS

Some modes of carrying out the invention are discussed below as preferred embodiments.

[First Embodiment]

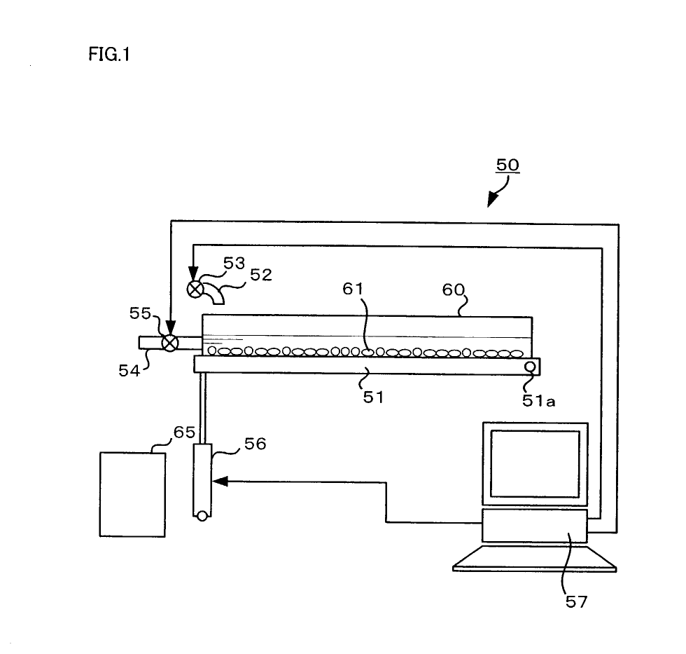

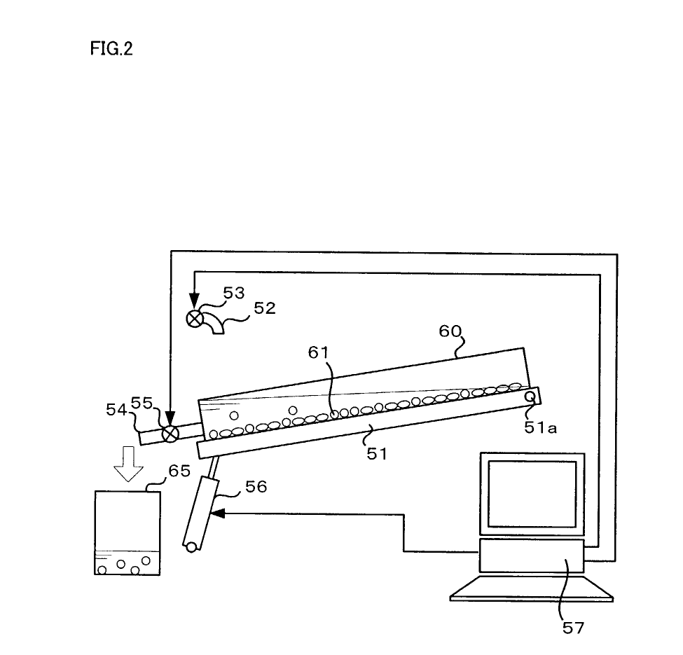

FIGS. 1 and 2 schematically illustrate the construction of a detached cells collection apparatus 50 in a first embodiment of the present invention. The detached cells collection apparatus 50 of this embodiment includes a support table 51, a culture chamber 60, and a control device 57, which functions as the time counter module and the classification-collection control module.

The culture chamber 60 is fixed and supported on the support table 51, which has a pivot 51a formed on one end thereof. The end of a rod of a power-driven cylinder 56 is attached to the other end of the support table 51. The power-driven cylinder 56 extends and contracts its rod in response to an instruction signal output from the control device 57. The support table 51 is in the horizontal orientation (see FIG. 1) in the extended position of the rod, and is in the inclined orientation (see FIG. 2) in the contracted position of the rod.

The culture chamber 60 includes anchorage-dependent cells (hereinafter simply referred to as cells) 61, which adhere to the bottom face of the culture chamber 60 and are proliferated in a culture medium to a specific state adequate for subculture. An injection pipe 52 is disposed in the vicinity of the culture chamber 60 to inject a trypsin solution as a release agent into the culture chamber 60. The injection pipe 52 has a solenoid valve 53 to open and close the injection pipe 52. An exhaust pipe 54 is attached to the culture chamber 60 to discharge the contents from the culture chamber 60. The exhaust pipe 54 has a solenoid valve 55 to open and close the exhaust pipe 54. A collection chamber 65 is set at the outlet of the exhaust pipe 54. The switching of each of the solenoid valves 53 and 55 is controlled in response to an instruction signal output from the control device 57. The injection pipe 52 with the solenoid valve 53 corresponds to the release agent injection module. The support table 51, the power-driven cylinder 56, the exhaust pipe 54 with the solenoid valve 55, and the collection chamber 65 correspond to the cell collection module.

The control device 57 has a known construction including a CPU, a ROM, a RAM, and a timer. The control device 57 uses the timer to count the time and outputs instruction signals to the respective solenoid valves 53 and 55 and the power-driven cylinder 56.

The trypsin processing time has some relationship to the proliferation potency as discussed below. An experiment was performed using a 75 cm² T flask (Nunc) as the culture chamber, human Keratinocyte taken from 0-year old human donor (preputia) (KK-4009, KURABO INDUSTRIES LTD) as the cell, and a serum-free medium for human Keratinocyte (Humedia-KG2, KURABO INDUSTRIES LTD) as the culture medium. The cell was cultured in the air containing 5% CO2 at the temperature of 37° C. to be confluent. A plurality of the culture chambers with such cells adhering to the bottom face were prepared. The procedure of the experiment took the culture medium off each of the multiple culture chambers, rinsed the culture chamber, and added a trypsin solution dropwise to the culture chamber. The procedure added trypsin inhibitor to the respective culture chambers after elapse of different time periods, 3 minutes, 6 minutes, 9 minutes, 12 minutes, and 15 minutes, since the dropwise addition of the trypsin solution (hereinafter this time period is referred to as the trypsin processing time) to inactivate trypsin. The procedure recovered the solution containing detached cells from the culture medium and centrifuged the recovered solution to collect the detached cells. The doubling time of the detached cells was measured as one index of the proliferation potency. The results of the experiment are shown in Table 1. As clearly understood from Table 1, the detached cells having the trypsin processing time of 3 minutes and that of 15 minutes have lower proliferation potency. In the case of the trypsin processing time of 3 minutes, it is presumed that a cell group having lowered adhesion power and proliferation potency by differentiation is detached and suspended preferentially. In the case of the trypsin processing time of 15 minutes, on the other hand, it is presumed that excess trypsin processing lowers the activity of the cells. In this manner, the trypsin processing time is closely related to the proliferation potency of the cells. The trypsin processing time may thus be regarded as one indication of the proliferation potency of the cells. Classification of detached cells according to the trypsin processing time enables divisional collection of cells having similar proliferation potency.

TABLE 1

| | Trypsin Processing Time tp[m] | | | | |
|---|---|---|---|---|---|
| | 3 | 6 | 9 | 12 | 15 |
| Doubling | 56.5 | 53.2 | 48.5 | 41.3 | 52.6 |
| Time td[h] | | | | | |

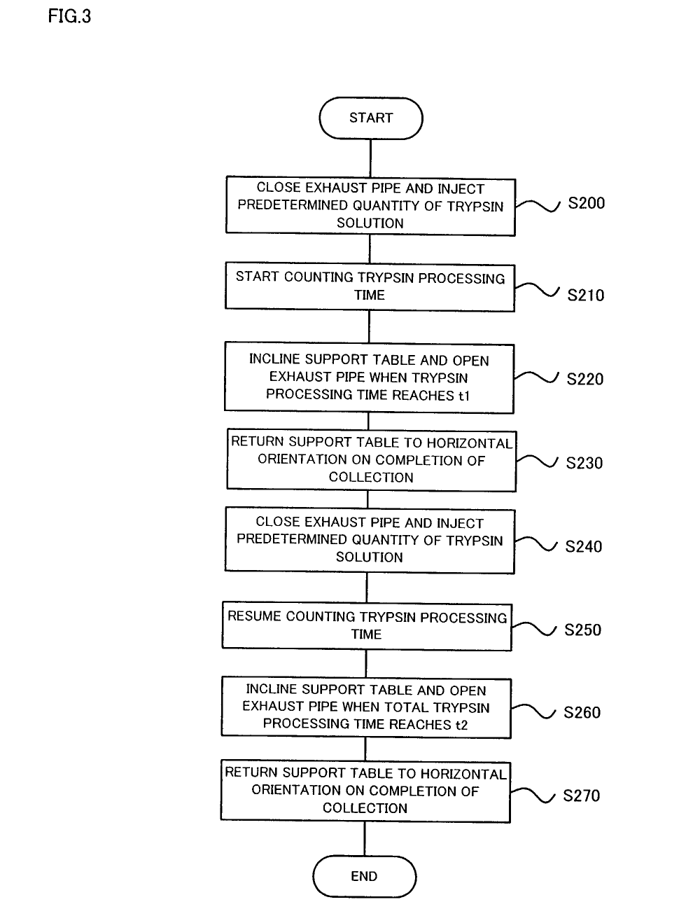

The detached cells collection apparatus 50 is operated according to the following process. In response to input of a detaching operation starting instruction through an operator's operation of a keyboard as an input device, the control device 57 reads and executes a corresponding cell detaching operation program stored in the internal memory. FIG. 3 is a flowchart showing the cell detaching operation program executed in the first embodiment. When this program starts, the control device 57 first activates the solenoid valve 55 to close the exhaust pipe 54, activates the solenoid valve 53 to open the injection pipe 52 and inject a predetermined quantity of the trypsin solution into the culture chamber 60, and then activates the solenoid valve 53 again to close the injection pipe 52 (step S200). Simultaneously with activation of the solenoid valve 53 to close the injection pipe 52, the timer included in the control device 57 starts counting the trypsin processing time (step S210). The culture medium is taken off and the culture chamber 60 is rinsed, prior to injection of the trypsin solution into the culture chamber 60. When the counted trypsin processing time reaches a preset time t1, the control device 57 contracts the rod of the power-driven cylinder 56 to incline the support table 51, and activates the solenoid valve 55 to open the exhaust pipe 54 and make the trypsin solution containing the detached cells 61 flown from the culture chamber 60 through the exhaust pipe 54 to the collection chamber 65 (step S220). The preset time t1 is a relatively short time period, for example, 3 minutes or 5 minutes. The cells 61 detached from the culture chamber 60 in an early stage are accordingly collected by the collection chamber 65 as an initial division.

On completion of such collection, the control device 57 extends the rod of the power-driven cylinder 56 to return the support table 51 to the horizontal orientation and sets a new collection chamber 65 in the detached cells collection apparatus 50 (step S230). Then the control device 57 activates the solenoid valve 55 to close the exhaust pipe 54, activates the solenoid valve 53 to open the injection pipe 52 and inject the predetermined quantity of the trypsin solution into the culture chamber 60, and activates the solenoid valve 53 again to close the injection pipe 52 (step S240). Simultaneously with activation of the solenoid valve 53 to close the injection pipe 52, the timer included in the control device 57 resumes counting the trypsin processing time (step S250). When the total trypsin processing time reaches a preset time t2, the control device 57 contracts the rod of the power-driven cylinder 56 to incline the support table 51, and activates the solenoid valve 55 to open the exhaust pipe 54 and make the trypsin solution containing the detached cells 61 flown from the culture chamber 60 through the exhaust pipe 54 to the collection chamber 65 (step S260). The preset time t2 is a relatively long time period, for example, 12 minutes or 15 minutes. The cells 61 detached from the culture chamber 60 after elapse of some time are accordingly collected by the collection chamber 65 as a main division. On completion of such collection, the control device 57 extends the rod of the power-driven cylinder 56 to return the support table 51 to the horizontal orientation and sets a new collection chamber 65 in the detached cells collection apparatus 50 (step S270). The control device 57 then exits from this program.

As described above, the detached cells collection apparatus 50 of the embodiment enables the cells detached from the bottom face of the culture chamber 60 by trypsin in the early stage to be separately collected from the cells detached from after elapse of some time. When there is a difference in proliferation potency between the cells detached from the culture chamber 60 by trypsin in the early stage and the cells detached from after elapse of some time, for example, as in the case of the cells taken from the 0-year-old human donor, the arrangement of the embodiment preferably ensures divisional collection of the cells according to the difference in proliferation potency.

In the flowchart of FIG. 3, the processing of steps S240 through S270 may be repeated a plurality of times according to the requirements. In this case, the time t2 at step S260 is changed to another time t3. When there is a difference in proliferation potency among the cells detached from the culture chamber 60 by trypsin in the early stage, the cells detached from after elapse of an intermediate time period, and the cells detached from after elapse of a longer time period, for example, as in the case of the cells taken from the 0-year-old human donor, this modified arrangement preferably ensures divisional collection of the cells according to the difference in proliferation potency.

[Second Embodiment]

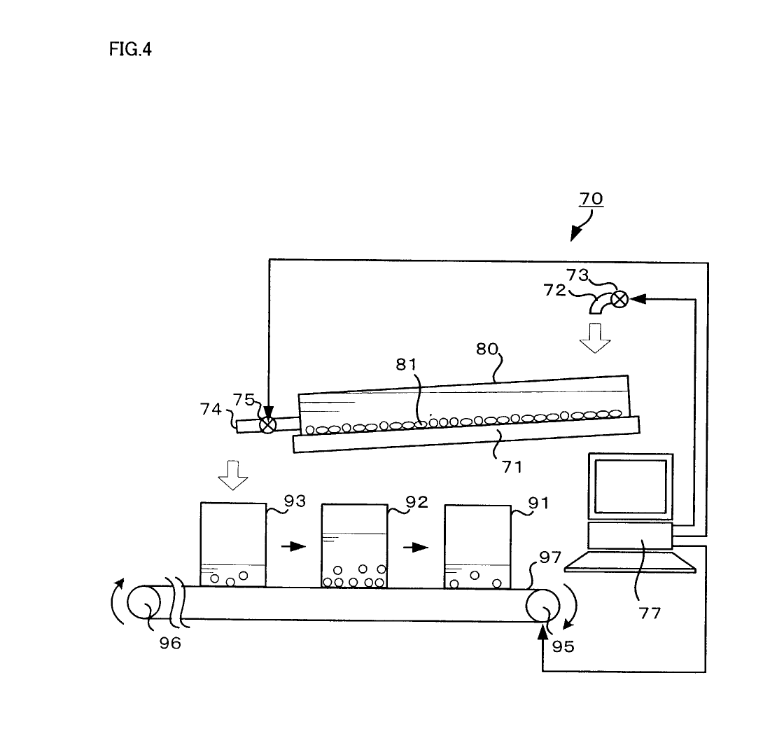

FIG. 4 schematically illustrates the construction of a detached cells collection apparatus 70 in a second embodiment of the present invention. The detached cells collection apparatus 70 of this embodiment includes a support table 71, a culture chamber 80, and a control device 77, which functions as the time counter module and the classification-collection control module.

The culture chamber 80 is fixed and supported in an inclined orientation on the support table 71.

The culture chamber 80 includes cells 81 that adhere to the bottom face of the culture chamber 80 and are proliferated in a culture medium to be confluent. An injection pipe 72 is disposed in the vicinity of the highest position of the culture chamber 80 supported by the support table 71 in the inclined orientation to inject a trypsin solution as the release agent into the culture chamber 80. The injection pipe 72 has a solenoid valve 73 to open and close the injection pipe 72. An exhaust pipe 74 is disposed in the vicinity of the lowest position of the culture chamber 80 to discharge the contents from the culture chamber 80. The exhaust pipe 74 has a solenoid valve 75 to open and close the exhaust pipe 74. The switching of each of the solenoid valves 73 and 75 is controlled in response to an instruction signal output from the control device 77.

Collection chambers 91 through 93 are mounted on a conveyor belt 97 spanned between a drive wheel 95 and a driven wheel 96, and are shifted with rotations of the drive wheel 95 in response to an instruction signal output from the control device 77 and a resulting movement of the conveyor belt 97. The collection chamber 91 is first located at a specific position corresponding to the outlet of the exhaust pipe 74. With elapse of time, the collection chamber 92 and the collection chamber 93 sequentially reach this specific position. The support table 71, the exhaust pipe 74 with the solenoid valve 75, the collection chambers 91 through 93, the drive wheel 95, the driven wheel 96, and the conveyor belt 97 correspond to the cell collection module.

The control device 77 has a known construction including a CPU, a ROM, a RAM, and a timer. The control device 77 uses the timer to count the time and outputs instruction signals to the respective solenoid valves 73 and 75 and the drive wheel 95.

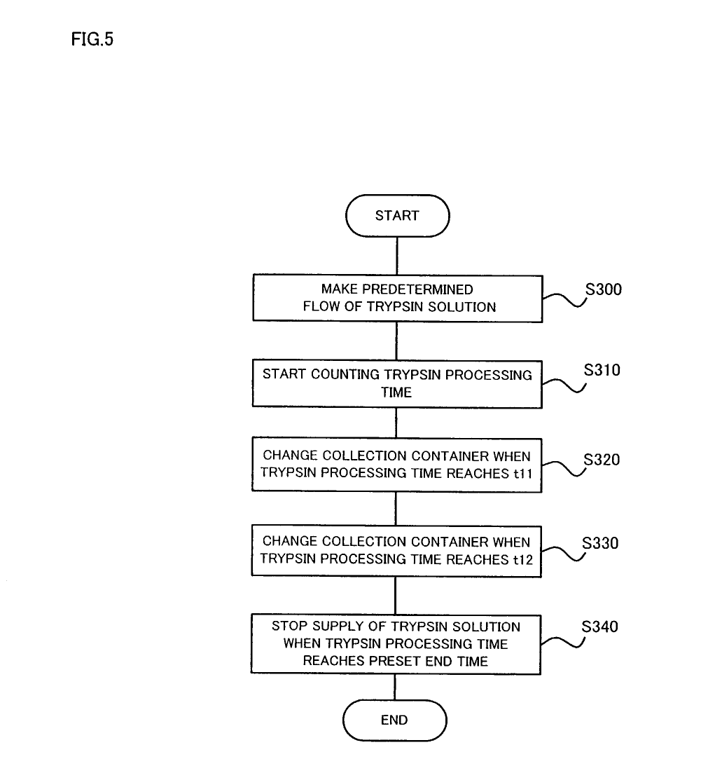

The detached cells collection apparatus 70 is operated according to the following process. In response to input of a detaching operation starting instruction through the operator's operation of the keyboard as the input device, the control device 77 reads and executes a corresponding cell detaching operation program stored in the internal memory. The culture medium is taken off and the culture chamber 80 is rinsed, prior to execution of this program. FIG. 5 is a flowchart showing the cell detaching operation program executed in the second embodiment. When this program starts, the control device 77 first regulates the openings of the solenoid valve 73 attached to the injection pipe 72 and the solenoid valve 75 attached to the exhaust pipe 74 to make a predetermined flow of the trypsin solution into the culture chamber 80 (step S300) and simultaneously activates the timer to start counting the trypsin processing time (step S310). Runoff from the culture chamber 80 is flown into the collection chamber 91 located at the specific position corresponding to the outlet of the exhaust pipe 74. The solenoid valves 73 and 75 are regulated to keep the substantially fixed quantity of the trypsin solution in the culture chamber 80 at step S300.

When the counted trypsin processing time reaches a preset time t11, the collection chamber 91 is changed to the next collection chamber 92 (step S320). The control device 77 outputs the instruction signal to the drive wheel 95 to rotate the drive wheel 95 and move the conveyor belt 97. The next collection chamber 92 accordingly reaches the specific position corresponding to the outlet of the exhaust pipe 74. The runoff from the culture chamber 80 is then flown into the collection chamber 92. Trypsin inhibitor is added to the previous collection chamber 91 to inactivate trypsin.

When the counted trypsin processing time reaches another preset time t12 (>t11), the collection chamber 92 is changed to the next collection chamber 93 (step S330). The control device 77 outputs the instruction signal to the drive wheel 95 to rotate the drive wheel 95 and move the conveyor belt 97. The next collection chamber 93 accordingly reaches the specific position corresponding to the outlet of the exhaust pipe 74. The runoff from the culture chamber 80 is then flown into the collection chamber 93. Trypsin inhibitor is added to the previous collection chamber 92 to inactivate trypsin.

When the counted trypsin processing time reaches a preset end time, the control device 77 activates the solenoid valve 73 to close the injection pipe 72 and stop the supply of the trypsin solution (step S340). After the culture chamber 80 is emptied out, the control device 77 activates the solenoid valve 75 to close the exhaust pipe 74. The control device 77 then exits from this program. Trypsin inhibitor is added to the collection chamber 93 to inactivate trypsin. The end time is preset empirically as the time required for detaching all the cells 81 adhering to the bottom face of the culture chamber 80 by the function of trypsin.

As described above, the detached cells collection apparatus 70 of the embodiment enables the cells detached from the bottom face of the culture chamber 80 by trypsin in the early stage to be separately collected from the cells detached from after elapse of some time. When there is a difference in proliferation potency between the cells detached from the culture chamber 80 by trypsin in the early stage, the cells detached from after elapse of an intermediate time period, and the cells detached from after elapse of a longer time period, for example, as in the case of the cells taken from the 0-year-old human donor, the arrangement of the embodiment preferably ensures divisional collection of the cells according to the difference in proliferation potency.

In the flowchart of FIG. 5, after step S340, the collection chamber may be changed to another collection chamber when the counted trypsin processing time reaches still another preset time, according to the requirements. This step may be repeated a plurality of times. This arrangement ensures segmentalized collection of cells according to the trypsin processing time.

The above embodiment is to be considered in all aspects as illustrative and not restrictive. There may be many modifications, change, and alterations without departing from the scope or spirit of the main characteristics of the present invention. All changes within the meaning and range of equivalency of the claims are therefore intended to be embraced therein.

What is claimed is:

1. A detached cells collection apparatus that collect anchorage-dependent cells, which are cultured in a culture chamber and are detached from said culture chamber, said detached cells collection apparatus comprising:
a time counter module that counts an elapsed time since injection of a release agent into said culture chamber with the anchorage-dependent cells adhering thereto;
a cell collection module that collects the anchorage-dependent cells in said culture chamber; and
a classification-collection control module that actuates said cell collection module to classify cells detached from said culture chamber by the release agent according to the elapsed time counted by said time counter module and collect the classified cells.

2. A detached cells collection apparatus in accordance with claim 1, said detached cells collection apparatus further comprising:
a release agent injection module that injects the release agent into said culture chamber,
wherein said classification-collection control module actuates said cell collection module according to the elapsed time to recover a substantially whole quantity of the release agent and thereby collect the cells detached from said culture chamber and included in the release agent, and subsequently actuates said release agent injection module to inject a new supply of the release agent to said culture chamber.

3. A detached cells collection apparatus in accordance with claim 2, wherein said classification-collection control module repeatedly carries out a process of actuating said cell collection module according to the elapsed time to recover a substantially whole quantity of the release agent and thereby collect the cells detached from said culture chamber and included in the release agent, and subsequently actuating said release agent injection module to inject a new supply of the release agent to said culture chamber.

4. A detached cells collection apparatus in accordance with claim 1, said detached cells collection apparatus further comprising:
a release agent injection module that injects the release agent into said culture chamber,
wherein said classification-collection control module actuates said cell collection module according to the elapsed time to recover part of the release agent and thereby collect the cells detached from said culture chamber and included in the release agent, and subsequently actuates said release agent injection module to inject a new supply of the release agent to said culture chamber.

5. A detached cells collection apparatus in accordance with claim 1, wherein said classification-collection control module actuates said cell collection module to collect the cells detached from said culture chamber as an initial division when the elapsed time counted by said time counter module reaches a preset initial time, and to collect the cells detached from said culture chamber after the preset initial time as a main division.

6. A detached cells collection apparatus in accordance with claim 1, wherein said classification-collection control module actuates said cell collection module to collect the cells detached from said culture chamber as an initial division when the elapsed time counted by said time counter module reaches a preset initial time, to collect the cells detached from said culture chamber as an intermediate division until the elapsed time reaches a preset latter time, and to collect the cells detached from said culture chamber after the preset later time as a last division.

7. A detached cells collection method for collecting anchorage-dependent cells, which are cultured in a culture chamber and are detached from said culture chamber, said detached cells collection method comprising:
a time counting step that counts an elapsed time since injection of a release agent into said culture chamber with the anchorage-dependent cells adhering thereto; and
a collection step that actuates a cell collection module, which is used for collecting cells in said culture chamber, to classify cells detached from said culture chamber by the release agent according to the elapsed time counted in said time counting step and collect the classified cells.

8. A detached cells collection method in accordance with claim 7, wherein said collection step actuates said cell collection module according to the elapsed time to recover a substantially whole quantity of the release agent and thereby collect the cells detached from said culture chamber and included in the release agent, and subsequently actuates a release agent injection module, which is used to inject the release agent into said culture chamber, to inject a new supply of the release agent to said culture chamber.

9. A detached cells collection method in accordance with claim 8, wherein said collection step repeatedly carries out a process of actuating said cell collection module according to the elapsed time to recover a substantially whole quantity of the release agent and thereby collect the cells detached from said culture chamber and included in the release agent, and subsequently actuating said release agent injection module to inject a new supply of the release agent to said culture chamber.

10. A detached cells collection method in accordance with claim 7, wherein said collection step actuates said cell collection module according to the elapsed time to recover part of the release agent and thereby collect the cells detached from said culture chamber and included in the release agent, and subsequently actuates a release agent injection module, which is used to inject the release agent into said culture chamber, to inject a new supply of the release agent to said culture chamber.

11. A detached cells collection method in accordance with claim 7, wherein said collection step actuates said cell collection module to collect the cells detached from said culture chamber as an initial division when the elapsed time counted by said time counter step reaches a preset initial time, and to collect the cells detached from said culture chamber after the preset initial time as a main division.

12. A detached cells collection method in accordance with claim 7, wherein said collection step actuates said cell collection module to collect the cells detached from said culture chamber as an initial division when the elapsed time counted by said time counter step reaches a preset initial time, to collect the cells detached from said culture chamber as an intermediate division until the elapsed time reaches a preset latter time, and to collect the cells detached from said culture chamber after the preset later time as a last division.

13. A detached cells collection method for collecting anchorage-dependent cells, which are cultured in a culture chamber and are detached from said culture chamber, said detached cells collection method comprising:
a step of actuating a cell collection module, which is used for collecting cells in said culture chamber, to classify cells detached from said culture chamber by a release agent according to an elapsed time since injection of the release agent into said culture chamber with the anchorage-dependent cells adhering thereto, and collect the classified cells.

14. A program that causes a computer to carry out an operation of collecting anchorage-dependent cells, which are cultured in a culture chamber and are detached from said culture chamber, said program causing the computer to attain:
a time counting function that counts an elapsed time since injection of a release agent into said culture chamber with the anchorage-dependent cells adhering thereto; and
a collection function that actuates a cell collection module, which is used for collecting cells in said culture chamber, to classify cells detached from said culture chamber by the release agent according to the elapsed time counted by said time counting function and collect the classified cells.

15. A program that causes a computer to carry out an operation of collecting anchorage-dependent cells, which are cultured in a culture chamber and are detached from said culture chamber, said program causing the computer to attain the function of:
actuating a cell collection module, which is used for collecting cells in said culture chamber, to classify cells detached from said culture chamber by a release agent according to an elapsed time since injection of the release agent into said culture chamber with the anchorage-dependent cells adhering thereto, and collect the classified cells.
